(19) United States
(12) Patent Application Publication
(10) Pub. No.: US 2016/0243202 A1
(43) Pub. Date: Aug. 25, 2016

(54) COMPOSITIONS AND METHODS FOR PREVENTION AND TREATMENT OF METABOLIC STEATOSIS AND STEATOHEPATITIS

(71) Applicant: International Nutrition Research Company, Luxembourg (LU)

(72) Inventor: Claude Vincent, Bordeaux (FR)

(21) Appl. No.: 14/661,526

(22) Filed: Mar. 18, 2015

(30) Foreign Application Priority Data
Feb. 24, 2015 (FR) 1551579

(51) Int. Cl. A61K 38/44; A61K 31/405; A61K 31/145; A61K 31/715; A61K 31/733; A61K 38/16; A61K 31/59; A61K 31/519; A61K 31/675; A61K 31/355; A61K 31/202; A61K 31/14; A61K 31/198; A61K 31/714

(52) U.S. Cl. CPC A61K 38/446; A61K 31/198; A61K 31/405; A61K 31/145; A61K 31/715; A61K 31/733; A61K 31/714; A61K 31/59; A61K 31/519; A61K 31/675; A61K 31/355; A61K 31/202; A61K 38/44; A61K 31/14; A61K 38/168; C12Y 115/01001; C12Y 111/01006

(57) ABSTRACT

The invention relates to an orally administered drug or medical nutrition composition for use in the prevention or treatment of metabolic steatosis and steatohepatitis of abdominally obese people with metabolic syndrome.

The composition comprises at least:
proteins and/or free amino adds,
fibers selected at least from acacia gum fibers and/or fructooligosaccharide fibers,
vitamins D, B6, B9, B12 and E,
omega 3 in the form of ALA,
glutathione,
SOD,
catalase,
choline.

### CROSS-REFERENCE TO RELATED APPLICATIONS

This application takes its priority from French patent application FR 1551579 filed Feb. 24, 2015, the entire disclosure of which is incorporated herein by reference.

### TECHNICAL FIELD OF THE INVENTION

The present invention relates to a drug or a medical nutrition composition which can be used in the prevention and treatment of metabolic steatosis and steatohepatitis.

### BACKGROUND

Steatosis is a pathological condition characterized by the presence of fat in the liver. It is an asymptomatic and reversible disease which is liable to evolve into steatohepatitis (characterized by a fibrosis), then into cirrhosis, and which increases the risk of hepatic cancers such as hepatocellular carcinoma.

There are two causes of steatosis: primary or metabolic steatosis which is caused by food and metabolic phenomena specific to the individual, and secondary steatosis which results from an “accidental” exogenous intoxication, for instance due to medicaments or chemical pollution.

Metabolic steatosis and steatohepatitis are the hepatic expression of metabolic syndrome. These are similar but less precise (eliminating secondary steatoses) terms for NAFLD (“Non-Alcoholic Fat Liver Disease”) and NASH (“Non-Alcoholic SteatoHepatitis”) for people with metabolic syndrome, that is to say people having at least 5% fat mass in the liver but with the presence of insulin resistance, inflammation and homocysteine in the liver (>12 ng/L). NAFLD is prevalent in 20 to 30% of the general population, and NASH in 2%. By contrast, metabolic steatosis and steatohepatitis are linked to weight and abdominal obesity with the presence of metabolic syndrome. Thus, 96% of patients undergoing bariatric surgery (having a BMI greater than 40) suffer from steatosis, and 25% from steatohepatitis. Likewise, it is known that 70% of patients with metabolic syndrome, 50% of obese people and 50% of diabetic people have steatohepatitis.

It is also known that the prevalence of steatohepatitis is increasing in all countries, even the most poor, and in the United States 6 million people have steatohepatitis, 600,000 of which have cirrhosis.

The 5-year survival rate for patients afflicted by metabolic steatohepatitis is 67%, and the 10-year survival rate is 38%.

It is difficult to differentiate between steatosis and steatohepatitis in the absence of an internationally recognized biochemical diagnostic test and because the “gold standard” remains hepatic biopsy which prohibits mass diagnosis. In addition, there is a grey area between benign steatosis and steatohepatitis where preventive measures must be taken in order to avoid the disease.

Besides biopsy, the markers are the scanner and MRI imaging and the ASAT, ALAT, γGT values, but above all the chronic inflammation markers TNFα and adiponectin and insulin resistance markers insulin levels, HOMA-IR, HOMA-S. Steatosis patients with metabolic syndrome should have a significant increase in ALAT and γGT.

At present, there is no solution to prevent and treat metabolic steatosis and steatohepatitis. The official recommendations are, in order:
restrict calories,
lose 3 to 5% weight,
reduce inflammation by 10%,
reduce hypertriglycericlemia, and
intake 300 IU of vitamin E.

These recommendations are too general to take account of the very particular physiology of steatohepatitis. In addition, 90-95% of people with metabolic syndrome fail at this regime within 3 years.

There is thus a need for a product which both facilitates weight loss, in particular facilitates the loss of visceral fat, and consolidates this over the long term by restoring the natural regulation cycles, by correcting the deregulations and deficiencies of people who are overweight and who have metabolic syndrome as the cause of the metabolic steatosis and steatohepatitis.

### SUMMARY OF INVENTION

The aim of the present invention is to meet this need.

To this end, the invention proposes a specific medical nutrition composition, comprising at least:
proteins and/or free amino acids,
fibers selected at least from acacia gum fibers and/or fructooligosaccharide fibers,
vitamins D, B6, B9, B12 and E,
omega 3 in the form of ALA,
glutathione,
SOD,
catalase,
choline,

the entirety of the amino acids present in the composition, stemming from the proteins and/or free amino acids, representing 40 to 60 g per 100 g of composition (active substances excluding any excipients), and the fibers representing between 2 and 10 g per 100 g of composition (active substances excluding any excipients).

Advantageously, such a composition can be used as a health product, in particular as a medical nutrition product or medical nutrition composition (“medical food”), for the prevention or treatment of metabolic steatosis and steatohepatitis of abdominally obese people with metabolic syndrome. The composition according to the invention can in particular be used to act on the various factors at the origin of these metabolic pathologies, in particular at the level of the digestive system and the liver.

### DETAILED DESCRIPTION

The invention will now be described in detail.

The invention therefore relates to an orally administered drug or medical nutrition composition for use in the prevention or treatment of metabolic steatosis and steatohepatitis of abdominally obese people with metabolic syndrome, comprising at least:
proteins and/or free amino acids,
fibers selected at least from acacia gum fibers and/or fructooligosaccharide fibers,
vitamins D, B6, B9, B12 and E,
omega 3 in the form of ALA,
glutathione,
SOD,
catalase,
choline,

the entirety of the amino acids present in the composition, stemming from the proteins and/or free amino acids, representing 40 to 90 g per 100 g of composition, and the fibers representing between 3 and 8 g per 100 g of composition.

The following abbreviations are used in this specification with their full names as follows
ALA ALPHA LINOLENIC ACID
AMPK ACTIVED PROTEIN KINASE
BAA BRANCHED AMINO ACID
EPA EICOSAPENTAENOIC ACID
FA FATTY ACID
FFA FREE FATTY ACID
GLP GLUCAGON LIKE PEPTIDE
IRS INSULIN RECEPTOR SUBSTRATE
LPS LIPOPOLYSACCHARIDES
LPA LYSOPHOSPHATIDIC ACID
MAO METHYLAMINE N OXIDE
NEFA NON ESTERIFIED FATTY ACID
NFKB NUCLEAR FACTOR
NO NITRIC OXIDE
PPAR PEROXISAME PROLIFERATOR ACTIVED RECEPTOR
PYY PEPTIDE YY
ROS REACTIVE OXYGEN SPECIES
SIBO SMALL INTESTIN BACTERIAL OVERGROWTH
SOD SUPEROXIDE DISMUTASE
TGF TRANSFORMING GROSS FACTOR
TLR TOLL LIKE RECEPTOR
TMAO TRIMETYLAMINE N OXIDE
TNF TRANSCRIPTION NUCLEAR FACTOR
AA ANTHRANILIC ACID
KA KYNURENIC ACID
QA QUINALDIC ACID
XA XANTUNERIC ACID

Within the meaning of the invention, the term drug is a medicine product or medical nutrition composition or medical nutrition product or “medical food” or foodstuffs intended for special medical purposes or dietary foods intended for special medical purposes means a foodstuff with a therapeutic purpose for prevention or treatment, used alone or in combination with other therapies. It is a food compound, responding to a particular clinical situation, which may form the sole or partial diet of the patients for which it is intended. In the context of the invention, it is in particular a product which is adapted to requirements and which corrects the metabolic disorders of abdominally obese people with metabolic syndrome.

Within the meaning of the invention, the term metabolic steatosis means a steatosis which has been caused by the presence of visceral fat for abdominally obese people.

Within the meaning of the invention, the term metabolic steatohepatitis means a steatohepatitis which has been caused by the presence of visceral fat for abdominally obese people.

Within the meaning of the invention, the term abdominally obese person means a person having a waist circumference greater than international standards (IDF 2005).

Within the meaning of the invention, the term metabolic syndrome means a syndrome which groups together at least three risk factors selected in particular from: waist circumference, blood sugar, HDL cholesterol, triglycerides and blood pressure, and which leads to metabolic and/or cardiovascular conditions.

Within the meaning of the invention, the term active substance means the active principles or ingredients which have a metabolic action, in opposition to excipients.

Throughout the present application, all percentages given in relation to the composition are given relative to the entirety of the active substances of the composition, that is to say the composition excluding any excipients.

Moreover, “100 g of composition” means 100 g of the entirety of the active substances of the composition, that is to say 100 g of the composition excluding any excipients.

According to one preferred embodiment, the composition according to the invention comprises at least the following amino acids: methionine, glycine, tryptophan, lysine, leucine, taurine and arginine. These amino acids are either free or contained in proteins. Methionine is preferably present in small quantity, whereas the other amino acids are present in larger quantity.

Preferably, the composition according to the invention comprises at least the following amino acids:
methionine, the methionine representing at most 2% by weight of the amino acids in the composition,
glycine, the glycine representing at least 3% by weight of the amino acids in the composition,
tryptophan, the tryptophan representing at least 2% by weight of the amino acids in the composition,
lysine, the lysine representing at least 6% by weight of the amino acids in the composition,
leucine, the leucine representing at least 7% by weight of the amino acids in the composition,
taurine, the taurine representing at least 2.5% by weight of the amino acids in the composition,
arginine, the arginine representing at least 5% by weight of the amino acids in the composition.

The composition may also comprise serine and cysteine.

Preferably, at least some of the amino acids are supplied by at least one plant protein and/or by at least one animal protein.

According to one particularly suitable embodiment, the plant protein(s) represent(s) between 30 and 90% by weight of the total weight of the proteins in the composition.

The plant protein(s) is/are preferably selected at least from pea proteins and/or say proteins and/or rice proteins, and each plant protein:
comprises methionine, in a quantity less than 2% by weight of the amino acids in the protein,
comprises glycine, in a quantity greater than 4% by weight of the amino acids in the protein, and has a methionine/glycine ratio of less than 1,
comprises branched amino acids, in a quantity less than 20% by weight of the amino acids in the protein,
comprises lysine, and has a lysine/arginine ratio of less than 2.

The animal protein(s) is/are preferably lactoserum having a degree of hydrolysis of at least 15%, preferably between 15 and 35%. Even more preferably, it is a lactoserum having a degree of hydrolysis of 25%, and the hydrolyzed lactoserum makes it possible to obtain:
tryptophan, representing at least 3% by weight of the amino acids in the lactoserum,
leucine, representing at least 10% by weight of the amino acids in the lactoserum,
lysine, representing at least 10% by weight of the amino acids in the lactoserum,
branched amino acids, representing at least 20% of the amino acids in the lactoserum,
a tryptophan/neutral amino acid ratio greater than 8% by weight of the amino acids in the lactoserum,
methionine, representing at most 3% by weight of the amino acids in the lactoserum.

According to one suitable embodiment, the composition comprises animal proteins and plant proteins, and the plant proteins represent between 30 and 90%, preferably between 60 and 80%, by weight of the proteins in the composition and the animal proteins represent between 10% and 70% by weight of the proteins in the composition, preferably between 20 and 40%.

Preferably, the composition is constituted and administered in such a way that the proteins in the composition represent approximately 50% of the daily protein intake of the patients to whom the composition is administered, taking account of the nutritional guidelines of 15% proteins in the traditional diet taken alongside the product according to the invention.

With regard to the fibers, the composition comprises at least acacia gum fibers and/or fructooligosaccharide fibers. Preferably, it comprises at least acacia gum fibers and fructooligosaccharide fibers.

The acacia gum preferably represents between 40 and 65% of the fibers in the composition. Similarly, the fructooligosaccharides preferably represent between 15 and 25% of the fibers in the composition.

Besides the basic constituents, the composition according to the invention may also contain one or more of the following compounds:
betaine,
calcium and/or zinc and/or chromium and/or selenium,
chlorogenic acid and/or a coffee extract,
native curcumin and/or soluble curcumin and/or omega 3 in the form of EPA
butylated hydroxytoluene.

If calcium is present in the composition according to the invention, it is preferably milk calcium obtained by precipitation from milk.

According to one particularly suitable embodiment, 100 g of composition (active substances excluding any excipients) comprises at least:
between 50 and 90 g of amino acids contained in proteins and/or in free form,
between 3 and 8 g of fibers,
between 5 and 12 μg of vitamin D.
between 1 and 3 mg of vitamin B6,
between 200 and 700 μg of vitamin B9,
between 2 and 6 μg of vitamin B12,
between 10 and 30 mg of vitamin E,
between 2 and 3.5 g of omega 3 in the form of ALA,
between 1000 and 2500 mg of glutathione,
between 100 and 250 mg of SOD,
between 200 and 450 mg of catalase,
between 1 and 2.5 g of choline.

Preferably, it comprises per 100 g of composition (active substances excluding any excipients):
between 20 and 70 g of plant proteins and between 9 and 55 g of animal proteins,
between 3 and 6.5 g of free amino acids in free form.

According to one variant, 100 g of composition (excluding excipients) also comprises:
between 10 and 30 mg of zinc, and/or
between 50 and 100 μg of chromium, and/or
between 60 and 120 μg of selenium, and/or
between 1 and 2.5 g of calcium, and/or
between 200 and 400 g of betaine, and/or
between 20 and 30 mg of butylated hydroxytoluene, and/or
between 1000 and 1300 mg of chlorogenic acid and/or coffee extract, and/or
between 600 and 1200 mg of native curcumin, and/or
between 600 and 1200 mg of soluble curcumin.

Preferably, 100 g of composition (active substances excluding excipients) according to the invention comprises at least:
between 20 and 70 g of pea and/or soy and/or rice proteins,
between 9 and 55 g of lactoserum hydrolysate having a degree of hydrolysis of from 15 to 35%,
between 3 and 6.5 g of free amino acids distributed as follows:
   between 1 and 1.5 g of lysine,
   between 2 and 3 g of taurine,
   between 0.5 and 3 g of tryptophan,
between 1500 and 8000 mg of fibers distributed as follows:
   between 1500 and 2600 mg of acacia gum,
   between 500 and 1500 mg of fructooligosaccharides,
   between 150 and 500 mg of wheat flour,
   between 600 and 2000 mg of pea bran,
between 5 and 12 μg of vitamin D,
between 1 and 3 mg of vitamin B6,
between 250 and 700 μg of vitamin B9,
between 2 and 6 μg of vitamin B12,
between 10 and 30 mg of vitamin E,
between 3 and 5 g of omega 3 in the form of ALA,
between 600 and 2500 mg of glutathione,
between 100 and 250 mg of SOD,
between 200 and 500 mg of catalase,
between 1 and 2.5 g of choline,
between 10 and 30 mg of zinc,
between 50 and 90 μg of chromium,
between 60 and 120 μg of selenium,
between 1 and 2.5 g of calcium,
between 200 and 400 mg of betaine,
between 20 and 40 mg of butylated hydroxytoluene,
between 1000 and 1300 mg of a mixture of chlorogenic acid and coffee extract,
between 600 and 1200 mg of native curcumin,
between 600 and 1200 mg of soluble curcumin.

The composition according to the invention can be obtained by a method as described below:
a first mixture is obtained by mixing the constituents in the following order: the animal and plant proteins as well as the free amino acids. The pH should be around 7 and should be stabilized at this level,
a second mixture is made using the cocktail of vitamins and minerals, then the fibers, then the ALA,
a third mixture is made using the antioxidants (glutathione, SOD, catalase), the betaine, the butylated hydroxytoluene, the chlorogenic acid and the coffee and then the two forms of curcumin,
add the two other mixtures to the first mixture while maintaining the pH at 7.

A powder is thus obtained which can be transformed into a tablet or liquid, or else used in its powder form in sachets, sticks, containers or capsules for example.

The composition according to the invention may be in any form suitable for oral administration. It may in particular be in the form of powder or granules, ready-to-drink beverages, bars or extruded products, the composition being supplemented with conventional excipients and fillers known to a person skilled in the art.

Preferably, it is in the form of powder or granules packaged in a sachet to be diluted in water.

Advantageously, the various constituents of the composition act in synergy to have an effect on metabolic steatosis and steatohepatitis. Thus, the composition according to the invention is an orally administered drug or medical nutrition composition intended to be used in the prevention or treatment of metabolic steatosis and steatohepatitis in abdominally obese people with metabolic syndrome.

Preferably, the composition is administered:
within the context of a calorie restriction corresponding approximately to 600 kcal less per day than the calculated total energy expenditure of the patients to whom the composition is administered,
as a supplement to daily physical activity of the patient corresponding to 30 minutes of walking or 10,000 steps.

The daily dose of composition according to the invention (dose of the mixture of active principles excluding excipients) is preferably between 60 and 120 g, preferably in two portions of 30 to 60 g, one taken in the morning at breakfast or as a snack at 11:00 am and one taken as a snack in the afternoon.

The physiology and physiopathology of metabolic steatosis and steatohepatitis are not yet fully known.

It originates from an overconsumption of calories, particularly an excess of fats (specifically saturated and monounsaturated fats) and/or fructose. A dysbiosis of the microbiota also sets in, with production of endogenous ethanol by Escherichia, of SIBO (small intestinal bacterial overgrowth), of LPS (by Escherichia coli) and LPA. It is also associated with an increase in FFAs and NEFAs (non-esterified fatty acids) and a reduction in butyrate and propionate, which leads to modulation of the NFκB signal of the inflammatory cytokines. Low-grade chronic inflammation and LPS (through activation of TLR5) will also cause insulin resistance.

Also noted are a reduction in the energy cycle through AMPKs and PPARs, a reduction in choline and methylation and an increase in homocysteine, a reduction in primary and secondary bile acids in the intestine, an increase in the phyla Actinobacteria of around 75% and Firmicutes of 25% and, depending on the excess weight or obesity, a reduction in Bifidobacteria, Lactobacillus and Bacteriodetes, resulting in an increase in the Firmicutes to Bacteriodetes ratio, a reduced functioning of the satiety neurotransmitters GLP1 and PYY, a reduction in serotonin 5HT which causes a drop in NO, a dysfunctioning of GLP1 by the receptor 5HTβ, activation of GLP2 and of the receptors TLR2 and TLR4 as a result of being attacked by an increasing number of ROSs and an increase in CD14 which causes the elasticity of the intestinal barrier to increase, allowing the passage of LPSs, LPAs, SIBOs, FFAs and NEFAs. A possible calcium deficiency increases this phenomenon by not capturing excess fats.

At the liver, steatosis is caused by:
the huge influx of LPSs, LPAs, SIBOs, FFAs, NEFAs and fructose which give rise to metabolic endotoxemia,
deregulation of the energy cycle through PPARs, caused in particular by FFAs,
a surge in β-oxidation requirements which is no longer able to keep up with the massive influx, causing cellular engorgement with peroxynitrite waste,
an increase in inflammation both in the intestine and in the liver, especially since in the meantime the fatty tissue has been able to develop and the macrophages give rise to inflammatory adipocytokines,
the catabolism of endogenous antioxidants such as glutathione, SOD and catalase by the inflammatory cytokines aggravates the problem,
triglyceride swelling of hepatocytes and of stellate cells,
disruption to cellular mitochondria and to the entire cell respiratory cycle.

Mitochondrial dysfunction accelerates deregulation of NFκB and the production of inflammatory cytokines. Inflammation inhibits the activity of insulin in the hepatocyte through the inhibition of IRS (insulin receptor substrate).

This system as a whole causes the liver to tip over into a permanent “de novo lipogenesis” (deregulation of AMPKs) which is self-sustaining. Moreover, over-activation of the mitochondria reduces AMPKs and the sensitivity to insulin introduces a lipotoxicity.

The transition from steatosis to the stage of steatohepatitis takes place by:
the reinforcement of insulin resistance and of inflammation, with profound dysfunction of the mitochondria,
a novo lipogenesis,
an increase in homocysteine by methylation of methionine and the usual deficiency in vitamins B6, B9 and B12,
an increase in fibrogenesis by TGF, characterizing steatohepatitis,
a reduction in omega 3,
an increase in the carnitine group and in the branched amino acids which are misused,
the action of glutathione peroxidase which accelerates the transformation into steatohepatitis.

Steatohepatitis is therefore marked, besides fibrosis, by a series of factors, in particular the increase in homocysteine and the damage inflicted by ROSs on the mitochondria which play a prominent role in the liver. Moreover, metabolic steatohepatitis is also linked to a dysfunction of the kynurenine pathway and indoleamine pathway of tryptophan. The tryptophan precursor of serotonin is used 80/90% in the intestine where it triggers the signal for the satiety neurotransmitters GLP1 and possibly GLP2 for the intestinal barrier. In addition, through TLR5, it produces NO and monosodium L-glutamate which increases the low thermogenesis in overweight people. After the intestine, the tryptophan is transported into the liver where it can be diverted into the kynurenine pathway by TNFα or cortisol. In a person with no inflammation and without metabolic syndrome, a small portion of 5% circulates in the plasma to supply the indoleamine pathway in the rest of the body for transforming the tryptophan into serotonin. The tryptophan (and not the serotonin) then crosses the blood-brain barrier if the ratio of tryptophan to neutral amino acids is greater than 7%, so as to give serotonin (mood) and melatonin (sleep).

The indoleamine pathway is the pathway for tryptophan to give 5HTP and then serotonin and finally 5HIAA (detectable in urine). The latter transition makes use of the enzyme MAO which then converts into TMAO, both of these being powerful oxidants. This phenomenon has been described as the hepatic oxidation engine in CMD (choline-methionine deficient) mice, which is not the case in humans and even less so in patients lacking tryptophan. This pathway requires PSP, a metabolite of vitamin B6, which is lacking in overweight people with metabolic syndrome.

The kynurenine pathway reserved for the liver for its part splits into 2 other pathways, the dysfunction of the first of which is governed by inflammatory cytokines and the dysfunction of the second of which is governed by cortisol from chronic stress. Therefore, due to inflammation, the kynurenine pathway becomes essential and produces xanthurenic acid XA, kynurenic acid KA, quinolinic acid QA, anthranilic acid AA and picolinic acid PA, which increase insulin resistance.

Advantageously, the present invention acts on the various mechanisms involved in metabolic steatosis and steatohepatitis.

The composition according to the invention is capable of acting on dysfunctions of the following systems:
homocysteine system,
mitochondrial respiratory system, and
kynurenine and indoleamine system of tryptophan/serotonin.

Advantageously, the presence of fibers, and in particular of acacia gum fibers and/or fructooligosaccharide fibers, makes it possible in particular:
to capture intestinal fats and to increase the butyrate and the propionate in the intestine,
to increase faecal evacuation of fats at the jejunum and duodenum, (+calcium+chlorogenic acid+coffee extract)
to increase the phyla Bifidobacteria longum and Lactobacillus so as to increase the fermentation of butyrate and propionate.

This has the result of activating GPL2 through TLR2 and TLR4 so as to reduce the intestinal production of LPSs, LPAs, FFAs, NEFAs, SIBOs and endogenous ethanol, and to reduce the membrane permeability of the intestinal barrier so as to reduce the passage of LPSs, LPAs, SIBOs, FAs and NEFAs.

The presence of vitamin B9 and of omega 3 ALA makes it possible to improve these effects. This is also the case when the composition contains zinc, selenium, purified EPA and tryptophan. Tryptophan makes it possible in particular to increase 5HT and thus the expression of the intestinal neurotransmitters GLP1, PYY so as to reinstate the feeling of satiety.

The presence of choline and of vitamins B6, B9 and B12 makes it possible to lower homocysteine.

The small quantity of methionine preferred according to the invention, as well as the presence of glycine, tryptophan, lysine, leucine, betaine and/or serine, also makes it possible to lower homocysteine.

Vitamin B6, and leucine when it is present, plays the role of mimicking physical activity which, preferably coupled with a slight calorie restriction, increases the AMPK and PPAR energy cycle.

Vitamin B6 also plays a role in reducing insulin resistance, in particular by lowering the XA, KA, QA, PA and AA of the kynurenine pathway. This is also the case for the acacia gum and omega 3 in the form of ALA and, when they are present in the composition, for the zinc, selenium, chromium, tryptophan, arginine, taurine, purified EPA and/or soluble curcumin.

Moreover, the hepatic enzymes SOD and catalase, as well as glutathione, vitamins D, B12 and E and choline, supplement the deficiency in endogenous antioxidants catabolized by inflammation, so as to combat metabolic endotoxemia in order to improve mitochondrial dysfunction by reducing the β-oxidation of the NEFAs and the production of TMAOs from MAOs. This effect is increased in the presence of zinc, chromium, selenium, soluble curcumin, butylated hydroxytoluene, chlorogenic acid or coffee extract, betaine and/or serine.

The presence of tryptophan is preferred because, besides the effects already mentioned, it makes it possible to contain the increase by the inflammatory cytokines of the kynurenine pathway which metabolizes tryptophan. It supplies the quantity necessary to increase the synthesis of serotonin via the indoleamine pathway to the detriment of the kynurenine pathway which is reduced.

This kynurenine pathway is also inhibited by the presence of choline, vitamin B6, B9 and B12, and by cysteine when the latter is present. These molecules also make it possible to replace methionine, the intake of which is low, so as to increase the production of glutathione, which strengthens the effect of the glutathione supplied by the composition.

According to another aspect, the omega 3 in the form of ALA makes it possible to regulate the NFκB signal of the inflammatory adipocytokines. This is also the case for the zinc, selenium, chromium, arginine, taurine, purified EPA and soluble curcumin when these elements are present in the composition.

Similarly, the omega 3 in the form of ALA, the vitamins B12 and D and the choline make it possible to lower the hepatic enzymes ASAT, ALAT, γGT, but also to increase HOMA-IR and HOMA-S. This effect can be reinforced in the presence of ALA and/or betaine.

Furthermore, vitamins D and E make it possible to regress the fibrosis which is one of the characteristics of steatohepatitis. The effect of these vitamins is accentuated in the presence of lysine and/or coffee extract.

Thus the composition, when it is administered to an abdominally obese patient with metabolic syndrome, makes it possible:
to limit the production of LPSs, LPAs, SIBOs, FFAs, NEFAs by reducing the intake of fats, particularly if the composition is taken within the context of calorie restriction, by a maximum evacuation of fats in the stools, by changing the phyla to eliminate endogenous ethanol and to increase butyrate and propionate,
to limit the passage of these molecules by reducing the elasticity of the intestinal membrane by regulating GLP2 through TLR2 and TLR4,
to regulate the methionine cycle by reducing the intake of methionine and by replacing it with glycine, serine and choline so as to produce glutathione,
to regulate the satiety receptors GLP1 and PYY,
to increase the production of AMPKs and PPARs,
to increase the antioxidants so as to restore beta-oxidation to a sufficient level,
to regulate hepatic mitochondrial function,
to regulate the kynurenine cycle TRP-KYN and the indoleamine cycle,
to lower intestinal and hepatic insulin resistance,
to regulate the signal NFκB so as to reduce the effect of the cytokines.

Similarly, the preferred variants of the composition according to the invention make it possible:
to increase tryptophan relative to neutral amino acids so as to cross the blood-brain barrier and improve mood, chronic stress and sleep,
to supply proteins with the minimum required quantity of methionine and branched amino acids (very numerous in NASH due to misuse), tryptophan, arginine, lysine, serine, threonine.

The efficacy of the invention can be measured in particular by measuring the reduction in tryptophan in the kynurenine pathway and the resumption of the indoleamine pathway by testing the urine for 5HIAA. It is also possible to check: hyaluronic acid, LPSs, MAOs, glutathione, homocysteine, methionine, choline, serine and cysteine, tryptophan and, BAAs, vitamins B6, B9, B12, D and finally malondialdehyde as antioxidant. Moreover, it is possible to measure the efficacy of the composition according to the invention by studying the microbiota comprising the conventional phyla of obesity, i.e. Bacteriodetes and Firmicutes, but also those of diabetes, Verrucomicrobia, and, for steatosis, Erysipelotrichi and Gammaproteobacteria, and finally, for butyrate, Lactobacillii and Bifidobacteria.

It should also be noted that there are varying degrees of steatohepatitis and fibrosis as shown in Tables 1 and 2 below:

TABLE 1

| | | LOBULAR | |
|---|---|---|---|
| DEGREE OF | % | INFLAMMA- | BALLOONING |
| STEATOHEPATITIS | STEATOSIS | TION | HEPATOCYTES |
| 1 | 5-33% | Mild | few |
| 2 | 34-66% | Moderate | many |
| 3 | >66% | Severe | |

TABLE 2

| DEGREE OF FIBROSIS | FIBROSIS |
|---|---|
| 1 | Mild perisinusoidal fibrosis |
| 2 | Moderate perisinusoidal fibrosis |
| 3 | Periportal fibrosis only |
| 4 | Periportal and perisinusoidal fibrosis |
| 5 | Bridging fibrosis |
| 6 | Cirrhosis |

The invention relates to all stages of steatohepatitis but only to degrees 1 to 4 of fibrosis. The invention will now be illustrated by examples.

### Example 1

The composition of example 1 consists of (excluding any excipients) as shown in Table 3 below:

TABLE 3

| | |
|---|---|
| Quantity per daily | Quantity per |
| ration of 2 portions | single portion |

PROTEINS

| | | | | |
|---|---|---|---|---|
| Pea proteins | 34 | g | 17 | g |
| Lactoserum hydrolysate DH 25% | 16 | g | 8 | g |
| TOTAL PROTEINS | 50 | g | 25 | g |

FREE AMINO ACIDS

| | | | | |
|---|---|---|---|---|
| Lysine | 1 | g | 0.5 | g |
| Taurine | 2 | g | 1 | g |
| Tryptophan | 1 | g | 0.50 | g |
| TOTAL FREE AAs | 4 | g | 2 | g |
| TOTAL AAs | 54 | g | 27 | g |

FIBERS

| | | | | |
|---|---|---|---|---|
| Acacia gum | 1600 | mg | 800 | mg |
| Fructooligosaccharides | 600 | mg | 300 | mg |
| Wheat flour (ALA) | 220 | mg | 110 | mg |
| Pea bran (protein) | 3240 | mg | 1620 | mg |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 300 | μg | 150 | μg |
| Vitamin B12 | 2.4 | μg | 1.2 | μg |
| Vitamin E | 12 | mg | 6 | mg |
| Zinc | 10 | mg | 5 | mg |
| Chromium | 40 | μg | 20 | μg |
| Selenium | 50 | μg | 25 | μg |
| Calcium | 1 | g | 0.50 | g |
| ALA (ω 3) | 1.5 | g | 0.75 | g |
| Choline | 1 | g | 0.5 | g |

ANTIOXIDANTS

| | | | | |
|---|---|---|---|---|
| Glutathione GSH | 500 | mg | 250 | mg |
| Catalase | 200 | mg | 100 | mg |
| SOD | 100 | mg | 50 | mg |
| Betaine (anhydrous) | 200 | mg | 100 | mg |
| Butylated hydroxytoluene | 20 | mg | 10 | mg |
| Chlorogenic acid/coffee extract | 800 | mg | 400 | mg |
| Native curcumin | 500 | mg | 250 | mg |
| Soluble curcumin | 500 | mg | 250 | mg |

Furthermore, the amino acids present in the composition (amino acids as constituents of proteins and free amino acids) include in particular the amino acids as presented in table 4 below:

TABLE 4

| | DAILY | PER | Percentage |
|---|---|---|---|
| | DOSE | PORTION | (of total |
| Amino acids (AA) | (g) | (g) | AAs) |
| LEUCINE | 4.70 | 2.35 | 8.57 |
| ISOLEUCINE | 2.58 | 1.29 | 4.8 |
| VALINE | 2.54 | 1.27 | 4 |
| Branched AAs | 9.82 | 4.91 | 18.2 |
| PHENYLALANINE + TYROSINE | 4.38 | 2.19 | 8.1 |
| TRYPTOPHAN | 1.84 | 0.92 | 3.4 |
| Aromatic AAs | 6.22 | 3.11 | 11.5 |
| Neutral amino acids (AAB + AAA) | 16.04 | 8.02 | 30.3 |
| ARGININIE | 3.30 | 1.65 | 6.1 |
| CYSTEINE | 0.66 | 0.33 | 1.2 |
| GLYCINE | 1.68 | 0.84 | 3.1 |
| GLUTAMINE | 8.64 | 4.32 | 15.7 |
| HISTIDINE | 1.26 | 0.63 | 2.3 |
| LYSINE | 5.22 | 2.61 | 9.7 |
| METHIONINE | 0.80 | 0.40 | 1.5 |
| SERINE | 2.54 | 1.27 | 4.7 |
| THREONINE | 2.18 | 1.09 | 4 |
| TAURINE | 2 | 1 | 3.7 |
| MISCELLANEOUS AAs | 28.2 | 14.1 | 52.4 |
| AAs described in the table | 44.3 | 22.2 | 82.1 |
| TOTAL AAs in the composition | 58 | 27 | 100 |

### Example 2

The composition of example 2 consists of (excluding any excipients) as shown in Table 5 below:

TABLE 5

| | |
|---|---|
| Quantity per daily | Quantity per |
| ration of 2 portions | single portion |

PROTEINS

| | | | | |
|---|---|---|---|---|
| Pea proteins | 30 | g | 15 | g |
| Lactoserum hydrolysate DH 25% | 4 | g | 2 | g |
| TOTAL PROTEINS | 34 | g | 17 | g |

FREE AMINO ACIDS

| | | | | |
|---|---|---|---|---|
| Taurine | 1 | g | 0.5 | g |
| Tryptophan | 0.5 | g | 0.25 | g |
| TOTAL FREE AAs | 1.5 | g | 0.75 | g |
| TOTAL AAs | 35.5 | g | 17.75 | g |

FIBERS

| | | | | |
|---|---|---|---|---|
| Acacia gum | 1600 | mg | 800 | mg |
| Fructooligosaccharides | 600 | mg | 300 | mg |
| Wheat flour (ALA) | 220 | mg | 110 | mg |
| Pea bran | 820 | mg | 410 | mg |
| TOTAL | 3020 | mg | 1510 | mg |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 300 | μg | 150 | μg |
| Vitamin B12 | 2.4 | μg | 1.2 | μg |
| Vitamin E | 12 | mg | 6 | mg |
| Zinc | 10 | mg | 5 | mg |
| Chromium | 40 | μg | 20 | μg |
| Selenium | 50 | μg | 25 | μg |
| ALA | 1.5 | g | 0.75 | g |
| Choline | 1 | g | 0.5 | g |

ANTIOXIDANTS

| | | | | |
|---|---|---|---|---|
| Glutathione | 1000 | mg | 500 | mg |
| Catalase | 200 | mg | 100 | mg |
| SOD | 100 | mg | 50 | mg |
| Native curcumin | 500 | mg | 250 | mg |
| Soluble curcumin | 500 | mg | 250 | mg |

Furthermore, the amino acids present in the composition (amino acids as constituents of proteins and free amino acids) include in particular the amino acids as presented in table 6 below:

TABLE 6

| | DAILY | PER | Percentage |
|---|---|---|---|
| | DOSE | PORTION | (of total |
| Amino acids (AAs) | (g) | (g) | AAs) |
| LEUCINE | 2.8 | 1.4 | 7.9 |
| ISOLEUCINE | 1.6 | 0.8 | 4.5 |
| VALINE | 1.8 | 0.9 | 5 |
| Branched AAs | 6.2 | 3.1 | 17.5 |
| PHENYLALANINE + TYROSINE | 3.2 | 1.6 | 9 |
| TRYPTOPHAN | 0.8 | 0.4 | 2.3 |
| Aromatic AAs | 4 | 2 | 11.3 |
| Neutral amino acids (AAB + AAA) | 10.2 | 5.1 | 28.7 |
| ARGININE | 3 | 1.5 | 8.4 |
| CYSTEINE | 0.4 | 0.2 | 1.1 |
| GLYCINE | 1.4 | 0.7 | 3.9 |
| GLUTAMINE | 5.8 | 2.9 | 16.3 |
| HISTIDINE | 0.8 | 0.4 | 2.2 |
| LYSINE | 2.4 | 1.2 | 6.8 |
| METHIONINE | 0.4 | 0.2 | 1.1 |
| SERINE | 1.8 | 0.9 | 5.1 |
| THREONINE | 1.4 | 0.7 | 3.9 |
| TAURINE | 1 | 0.5 | 2.7 |
| MISCELLANEOUS AAs | 18.4 | 9.2 | 51.8 |
| AAs described in the table | 28.6 | 14.3 | 80.6 |
| TOTAL AAs in the composition | 35.5 | 17.75 | 100 |

### Example 3

The composition of example 3 consists of (excluding any excipients) as shown in Table 7 below:

TABLE 7

| | |
|---|---|
| Quantity per daily | Quantity per |
| ration of 2 portions | single portion |

PROTEINS

| | | | | |
|---|---|---|---|---|
| Rice proteins | 50 | g | 25 | g |
| Lactoserum hydrolysate DH 25% | 10 | g | 5 | g |
| TOTAL PROTEINS | 60 | g | 30 | g |

FREE AMINO ACIDS

| | | | | |
|---|---|---|---|---|
| Lysine | 1 | g | 0.5 | g |
| Taurine | 2 | g | 1 | g |
| Tryptophan | 1 | g | 0.5 | g |
| TOTAL FREE AAs | 4 | g | 2 | g |
| TOTAL AAs | 64 | g | 32 | g |

FIBERS

| | | | | |
|---|---|---|---|---|
| Acacia gum | 1400 | mg | 700 | mg |
| Fructooligosaccharides | 400 | mg | 200 | mg |
| What flour (ALA) | 220 | mg | 110 | mg |
| Rice bran | 1700 | mg | 850 | mg |
| TOTAL | 3720 | mg | 1860 | mg |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 300 | μg | 150 | μg |
| Vitamin B12 | 2.4 | μg | 1.2 | μg |
| Vitamin E | 12 | mg | 6 | mg |
| Zinc | 1.5 | g | 0.75 | g |
| Chromium | 1 | g | 0.5 | g |
| Selenium | 5 | μg | 2.5 | μg |
| ALA | 1.4 | mg | 0.7 | mg |
| Choline | 300 | μg | 150 | μg |

ANTIOXIDANTS

| | | | | |
|---|---|---|---|---|
| Glutathione | 1000 | mg | 500 | mg |
| Catalase | 200 | mg | 100 | mg |
| SOD | 100 | mg | 50 | mg |
| Native curcumin | 500 | mg | 250 | mg |
| Soluble curcumin | 500 | mg | 250 | mg |

Furthermore, the amino acids present in the composition (amino acids as constituents of proteins and free amino acids) include in particular the amino acids as presented in table 8 below:

TABLE 8

| | DAILY | PER | Percentage |
|---|---|---|---|
| | DOSE | PORTION | (of total |
| Amino acids (AAs) | (g) | (g) | AAs) |
| LEUCINE | 5.0 | 2.5 | 7.8 |
| ISOLEUCINE | 2.6 | 1.3 | 4.1 |
| VALINE | 3 | 1.5 | 4.7 |
| Branched AAs | 10.8 | 5.3 | 16.6 |
| PHENYLALANINE + TYROSINE | 5.6 | 2.8 | 8.7 |
| TRYPTOPHAN | 1.6 | 0.8 | 2.5 |
| Aromatic AAs | 7.2 | 3.6 | 11.3 |
| Neutral amino acids (AAB + AAA) | 18 | 9 | 27.9 |
| ARGININE | 5.2 | 2.6 | 8.1 |
| CYSTEINE | 0.6 | 0.3 | 0.9 |
| GLYCINE | 2.4 | 1.2 | 3.7 |
| GLUTAMINE | 10 | 5 | 15.6 |
| HISTIDINE | 1.5 | 0.7 | 2.2 |
| LYSINE | 4.4 | 2.2 | 6.9 |
| METHIONINE | 0.6 | 0.3 | 0.9 |
| SERINE | 3.2 | 1.6 | 5 |
| THREONINE | 2.4 | 1.2 | 3.7 |
| TAURINE | 2 | 1 | 3.1 |
| MISCELLANEOUS AAs | 32.2 | 16.1 | 50.3 |
| AAs described in the table | 50.2 | 25.1 | 78.4 |
| TOTAL AAs in the composition | 64 | 32 | 100 |

### Example 4

The composition of example 4 consists of (excluding any excipients) as shown in Table 9 below:

TABLE 9

| | |
|---|---|
| Quantity per daily | Quantity per |
| ration of 2 portions | single portion |

PROTEINS

| | | | | |
|---|---|---|---|---|
| Pea proteins | 40 | g | 20 | g |
| Lactoserum hydrolysate DH 25% | 20 | g | 10 | g |
| TOTAL PROTEINS | 60 | g | 30 | g |

FREE AMINO ACIDS

| | | | | |
|---|---|---|---|---|
| Lysine | 1 | g | 0.5 | g |
| Taurine | 2 | g | 1.0 | g |
| Tryptophan | 1.6 | g | 0.8 | g |
| TOTAL FREE AAs | 4.6 | g | 2.3 | g |
| TOTAL AAs | 64.6 | g | 32.3 | g |

FIBERS

| | | | | |
|---|---|---|---|---|
| Acacia gum | 1600 | mg | 800 | mg |
| Fructooligosaccharides | 600 | mg | 300 | mg |
| Wheat flour (ALA) | 220 | mg | 110 | mg |
| Pea bran | 960 | mg | 480 | mg |
| TOTAL | 3380 | mg | 1690 | mg |

MICRONUTRIENTS

| | | | | |
|---|---|---|---|---|
| Vitamin D | 5 | μg | 2.5 | μg |
| Vitamin B6 | 1.4 | mg | 0.7 | mg |
| Vitamin B9 | 300 | μg | 150 | μg |
| Vitamin B12 | 2.4 | μg | 1.2 | μg |
| Vitamin E | 12 | mg | 6 | mg |
| Zinc | 10 | mg | 5 | mg |
| Chromium | 40 | μg | 20 | μg |
| Selenium | 50 | μg | 25 | μg |
| ALA | 1 | g | 0.5 | g |
| Choline | 1.5 | g | 0.75 | g |

ANTIOXIDANTS

| | | | | |
|---|---|---|---|---|
| Glutathione | 1000 | mg | 500 | mg |
| Catalase | 200 | mg | 100 | mg |
| SOD | 100 | mg | 50 | mg |
| Native curcumin | 500 | mg | 250 | mg |
| Soluble curcumin | 500 | mg | 250 | mg |

Furthermore, the amino acids present in the composition (amino acids as constituents of proteins and free amino acids) include in particular the amino acids as presented in table 10 below:

TABLE 10

| | DAILY | PER | Percentage |
|---|---|---|---|
| | DOSE | PORTION | (of total |
| Amino acids (AAs) | (g) | (g) | AAs) |
| LEUCINE | 5.8 | 2.9 | 8.9 |
| ISOLEUCINE | 3.2 | 1.6 | 4.9 |
| VALINE | 3 | 1.5 | 4.6 |
| Branched AAs | 12 | 6 | 18.4 |
| PHENYLALANINE + TYROSINE | 5.2 | 2.6 | 8 |
| TRYPTOPHAN | 2.6 | 1.3 | 4 |
| Aromatic AAs | 7.8 | 3.9 | 12 |
| Neutral amino acids (AAB + AAA) | 17.6 | 9.9 | 30.4 |
| ARGININE | 3.8 | 1.9 | 5.9 |
| CYSTEINE | 1 | 0.5 | 1.5 |
| GLYCINE | 2 | 1 | 3.1 |
| GLUTAMINE | 10.4 | 5.2 | 16.1 |
| HISTIDINE | 1.6 | 0.8 | 2.5 |
| LYSINE | 6 | 3.0 | 9.3 |
| METHIONINE | 1 | 0.5 | 1.5 |
| SERINE | 3.2 | 1.6 | 4.9 |
| THREONINE | 2.6 | 1.3 | 4 |
| TAURINE | 2 | 1 | 3.1 |
| MISCELLANEOUS AAs | 33.6 | 16.8 | 52.0 |
| AAs described in the table | 51.2 | 25.6 | 79.2 |
| TOTAL AAs in the composition | 64.6 | 32.3 | 100 |

### Example 5

The composition of example 5 consists of (excluding any excipients) as shown in Table 11 below:

TABLE 11

| | |
|---|---|
| Quantity per daily | Quantity per |
| ration of 2 portions | single portion |

PROTEINS

| | | | | |
|---|---|---|---|---|
| Pea proteins | 20 | g | 10 | g |
| Lactoserum hydrolysate DH 25% | 40 | g | 20 | g |
| TOTAL PROTEINS | 60 | g | 30 | g |

FREE AMINO ACIDS

| | | | | |
|---|---|---|---|---|
| Taurine | 2 | g | 1 | g |
| Tryptophan | 0.6 | g | 0.3 | g |
| TOTAL FREE AAs | 2.6 | g | 1.3 | g |
| TOTAL AAs | 62.6 | g | 31.3 | g |

FIBERS

| | | | | |
|---|---|---|---|---|
| Acacia gum | 1600 | mg | 800 | mg |
| Fructooligosaccharides | 600 | mg | 300 | mg |
| Wheat flour (ALA) | 220 | mg | 110 | mg |
| Pea bran | 480 | mg | 240 | mg |
| TOTAL | 2900 | mg | 1450 | mg |

MICRONUTRIENTS

| | | | | |
|---|---|---|---|---|
| Vitamin D | 6 | μg | 3 | μg |
| Vitamin B6 | 2 | mg | 1 | mg |
| Vitamin B9 | 400 | μg | 200 | μg |
| Vitamin B12 | 4.0 | μg | 2.0 | μg |
| Vitamin E | 20 | mg | 10 | mg |
| Zinc | 20 | mg | 10 | mg |
| Chromium | 60 | μg | 30 | μg |
| Selenium | 50 | μg | 25 | μg |
| ALA | 1.6 | g | 0.8 | g |
| Choline | 2 | g | 1 | g |

ANTIOXIDANTS

| | | | | |
|---|---|---|---|---|
| Glutathione | 1000 | mg | 500 | mg |
| Catalase | 200 | mg | 100 | mg |
| SOD | 100 | mg | 50 | mg |
| Native curcumin | 500 | mg | 250 | mg |
| Soluble curcumin | 500 | mg | 250 | mg |

Furthermore, the amino acids present in the composition (amino acids as constituents of proteins and free amino acids) include in particular the amino acids as presented in table 12 below:

TABLE 12

| | DAILY | PER | Percentage |
|---|---|---|---|
| | DOSE | PORTION | (of total |
| Amino acids (AAs) | (g) | (g) | AAs) |
| LEUCINE | 6.4 | 3.2 | 10.2 |
| ISOLEUCINE | 3.6 | 1.8 | 5.7 |
| VALINE | 3 | 1.5 | 4.8 |
| Branched AAs | 13.4 | 6.5 | 20.7 |
| PHENYLALANINE + TYROSINE | 5 | 2.5 | 8.0 |
| TRYPTOPHAN | 1.8 | 0.9 | 2.9 |
| Aromatic AAs | 6.4 | 3.4 | 10.9 |
| Neutral amino acids (AAB + AAA) | 19.8 | 9.9 | 32.1 |
| ARGININE | 3 | 1.6 | 5 |
| CYSTEINE | 1 | 0.5 | 1.5 |
| GLYCINE | 1.6 | 0.8 | 2.6 |
| GLUTAMINE | 6 | 3.0 | 9.6 |
| HISTIDINE | 1.8 | 0.9 | 2.9 |
| LYSINE | 5.8 | 2.9 | 9.3 |
| METHIONINE | 1.4 | 0.7 | 2 |
| SERINE | 3 | 1.5 | 4.6 |
| THREONINE | 2.8 | 1.4 | 4.5 |
| TAURINE | 2 | 1 | 3.2 |
| MISCELLANEOUS AAs | 28.4 | 14.3 | 45.2 |
| AAs described in the table | 48.2 | 24.1 | 77.1 |
| TOTAL AAs in the composition | 64.6 | 31.3 | 100 |

What is claimed is:

1. An orally administered drug or medical nutrition composition for prevention and treatment of metabolic steatosis and steatohepatitis of abdominally obese people with metabolic syndrome, comprising at least:
at least one of proteins and free amino acids,
fibers selected at least from acacia gum fibers and fructooligosaccharide fibers,
vitamins D, B6, B9, B12 and E,
omega 3 in the form of ALA (ALPHA LINOLENIC ACID),
glutathione,
SOD (SUPEROXIDE DISMUTASE),
catalase,
choline,
the entirety of the amino acids present in the composition, stemming from the proteins and free amino acids, representing 40 to 90 g per 100 g of composition (excluding any excipients), and the fibers representing between 3 and 8 g per 100 g of composition (excluding any excipients).

2. The composition of claim 1, wherein the composition comprises at least the following amino acids: methionine, glycine, tryptophan, lysine, leucine, taurine and arginine.

3. The composition of claim 1, wherein the composition comprises at least the following amino acids:
methionine, the methionine representing at most 2% by weight of the amino acids in the composition,
glycine, the glycine representing at least 3% by weight of the amino acids in the composition,
tryptophan, the tryptophan representing at least 2% by weight of the amino acids in the composition,
lysine, the lysine representing at least 6% by weight of the amino acids in the composition,
leucine, the leucine representing at least 7% by weight of the amino acids in the composition,
taurine, the taurine representing at least 2.5% by weight of the amino acids in the composition,
arginine, the arginine representing at least 5% by weight of the amino acids in the composition.

4. The composition of claim 1, wherein the composition comprises serine and cysteine.

5. The composition of claim 1, wherein the composition comprises at least one plant protein.

6. The composition of claim 5, wherein the plant protein represents between 30 and 90% by weight of the total weight of the proteins in the composition.

7. The composition of claim 5, wherein the plant protein is selected from the group consisting pea proteins, soy proteins and rice proteins, and the plant protein:
comprises methionine, in a quantity less than 2% by weight of the amino acids in the protein,
comprises glycine, in a quantity greater than 4% by weight of the amino acids in the protein, and has a methionine/glycine ratio of less than 1,
comprises branched amino acids, in a quantity less than 20% by weight of the amino acids in the protein,
comprises lysine, and has a lysine/arginine ratio of less than 2.

8. The composition of claim 1, wherein the composition comprises at least one animal protein.

9. The composition of claim 1, wherein the composition comprises lactoserum having a degree of hydrolysis of 25%.

10. The composition of claim 9, wherein hydrolyzed lactoserum makes it possible to obtain:
tryptophan, representing at least 3% by weight of the amino acids in the lactoserum,
leucine, representing at least 10% by weight of the amino acids in the lactoserum,
lysine, representing at least 10% by weight of the amino acids in the lactoserum,
branched amino acids, representing at least 20% of the amino acids in the lactoserum,
a tryptophan/neutral amino add ratio greater than 8% by weight of the amino adds in the lactoserum,
methionine, representing at least 3% by weight of the amino acids in the lactoserum.

11. The composition of claim 1, wherein the composition comprises at least one of the following compounds:
betaine,
calcium and/or zinc and/or chromium and/or selenium,
chlorogenic acid and/or a coffee extract,
native curcumin and/or soluble curcumin and/or omega 3 in the form of EPA (EICOSAPENTAENOIC ACID)
butylated hydroxytoluene.

12. The composition of claim 11, wherein the calcium is a milk calcium obtained by precipitation from milk.

13. The composition of claim 1, wherein the composition comprises animal proteins and plant proteins, and in that the plant proteins represent between 30 and 90% by weight of the proteins in the composition and the animal proteins represent between 10% and 70% by weight of the proteins in the composition.

14. The composition of claim 1, wherein the acacia gum represents between 40 and 65% of the fibers in the composition.

15. The composition of claim 1, wherein the fructooligosaccharides represent between 15 and 25% of the fibers in the composition.

16. The composition of claim 1, wherein the composition is administered within the context of a calorie restriction corresponding approximately to 600 kcal less per day than the calculated total energy expenditure of the patients to whom the composition is administered.

17. The composition of claim 1, wherein the composition is administered as a supplement to daily physical activity of a patient, wherein the daily physical activity corresponds to 30 minutes of walking or 10,000 steps.

18. The composition of claim 1, wherein 100 g of the composition (excluding any excipients) comprises at least:
between 50 and 90 g of amino acids contained in proteins and/or in free form,
between 3 and 8 g of fibers,
between 5 and 12 μg of vitamin D,
between 1 and 3 mg of vitamin B6,
between 200 and 700 μg of vitamin B9,
between 2 and 6 μg of vitamin B12,
between 10 and 30 mg of vitamin E,
between 2 and 3.5 g of omega 3 in the form of ALA,
between 1000 and 2500 mg of glutathione,
between 100 and 250 mg of SOD,
between 200 and 450 mg of catalase,
between 1 and 2.5 g of choline.

19. The composition of claim 18, wherein 100 g the composition (excluding any excipients) comprises between 20 and 70 g of plant proteins and between 9 and 55 g of animal proteins.

20. The composition of claim 18 wherein 100 g of the composition (excluding any excipients) comprises between 3 and 6.5 g of amino acids in free form.

21. The composition claim 18 wherein 100 g of the composition (excluding any excipients) further comprises:
between 10 and 30 mg of zinc, and/or
between 50 and 100 μg of chromium, and/or
between 60 and 120 μg of selenium, and/or
between 1 and 2.5 g of calcium, and/or
between 200 and 400 g of betaine, and/or
between 20 and 30 mg of butylated hydroxytoluene, and/or
between 1000 and 1300 mg of chlorogenic acid and/or coffee extract, and/or
between 600 and 1200 mg of native curcumin, and/or
between 600 and 1200 mg of soluble curcumin.

22. The composition of claim 1 wherein 100 g of the composition (excluding any excipients) comprises at least:
between 20 and 70 g of pea and/or soy and/or rice proteins,
between 9 and 55 g of lactoserum hydrolysate having a degree of hydrolysis between 15 and 35%,
between 3 and 6.5 g of free amino acids distributed as follows:
between 1 and 1.5 g of lysine,
between 2 and 3 g of taurine,
between 0.5 and 3 g of tryptophan,
between 1500 and 8000 mg of fibers distributed as follows:
between 1500 and 2600 mg of acacia gum,
between 500 and 1500 mg of fructooligosaccharides,
between 150 and 500 mg of wheat flour,
between 600 and 2000 mg of pea bran,
between 5 and 12 μg of vitamin D,
between 1 and 3 mg of vitamin B6,
between 250 and 700 μg of vitamin B9,
between 2 and 6 μg of vitamin B12,
between 10 and 30 mg of vitamin E,
between 3 and 5 g of omega 3 in the form of ALA,
between 600 and 2500 mg of glutathione,
between 100 and 250 mg of SOD,
between 200 and 500 mg of catalase,
between 1 and 2.5 g of choline,
between 10 and 30 mg of zinc,
between 50 and 90 μg of chromium,
between 60 and 120 μg of selenium,
between 1 and 2.5 g of calcium,
between 200 and 400 mg of betaine,
between 20 and 40 mg of butylated hydroxytoluene,
between 1000 and 1300 mg of chlorogenic acid and/or coffee extract,
between 600 and 1200 mg of native curcumin,
between 600 and 1200 mg of soluble curcumin.
The composition of claim 18, wherein 100 g the composition (excluding any excipients) comprises between 20 and 70 g of plant proteins and between 9 and 55 g of animal proteins.

23. A method of treatment for an abdominally obese patient with metabolic syndrome in the need of treatment for at least one of the following conditions: metabolic steatosis and steatohepatitis, wherein the method comprises administering to the patient the composition of claim 1.
